(19) United States
(12) Patent Application Publication
(10) Pub. No.: US 2010/0266642 A1
(43) Pub. Date: Oct. 21, 2010

(54) MODIFIED CELLS FOR TARGETED CELL TRAFFICKING AND USES THEREOF

(75) Inventors: Susan Langer, Newton, MA (US); Jeff Hrkach, Lexington, MA (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: BIND BIOSCIENCES, INC., Cambridge, MA (US)

(21) Appl. No.: 12/708,191

(22) Filed: Feb. 18, 2010

Related U.S. Application Data
(60) Provisional application No. 61/154,250, filed on Feb. 20, 2009.

(51) Int. Cl. A61K 39/00; A61P 35/00; A61P 35/02; C12N 5/00; A61K 35/12

(52) U.S. Cl. 424/277.1; 435/325; 424/93.1

(57) ABSTRACT

This application provides methods of making modified cells containing pre-functionalized poly(ethylene glycol) (PEG) and/or other pre-functionalized polymers are provided. The modified cells produced according to the disclosed methods as well as their use in the treatment of various diseases are also provided.

### CROSS-REFERENCE TO RELATED APPLICATION

This application claims the benefit of U.S. Provisional Patent Application 61/154,250, filed Feb. 20, 2009, the disclosure of which is hereby incorporated by reference in its entirety.

### BACKGROUND OF THE INVENTION

Targeting therapeutic agent to a desired site of action (e.g., targeted to a particular tissue or cell type or targeted to a specific diseased tissue but not normal tissue) is desirable because it reduces the amount of a drug present systemically in a subject and can result in reduced side effects during treatment of a disease or condition, such as cancer. This is particularly important when treating a condition such as cancer where it is desirable that a therapeutic dose of the drug is delivered to target cells without adversely affecting surrounding tissue. Accordingly, a need exists to develop delivery systems which can deliver therapeutic levels of drug to treat diseases such as cancer, while also reducing patient side effects.

### BRIEF SUMMARY OF THE INVENTION

This application provides methods of making modified cells and related compositions using pre-functionalized poly(ethylene glycol)(also referred to as PEG) and/or a second biocompatible and biodegradable hydrophobic polymer (e.g., a poly(ester)). The poly(ethylene glycol) is hetero-bifunctional with a targeting moiety (agent) covalently bound to its α terminus and a functional group (e.g., a hydroxyl group) present on its ω terminus. Modified cells produced according to the disclosed methods and their use in the treatment of various diseases and disorders is also provided.

### DETAILED DISCLOSURE OF THE INVENTION

This application provides methods of making modified cells using pre-functionalized poly(ethylene glycol)(also referred to as PEG) and/or pre-functionalized biocompatible and biodegradable hydrophobic polymer (sometimes referred to as a pre-functionalized polymer block herein). Modified cells can also be made using PEG and/or biocompatible and biodegradable hydrophobic polymer blocks that have not been pre-functionalized. The PEG and/or biocompatible and biodegradable hydrophobic polymer block can be functionalized with a targeting agent after attachment of a cell.

As used herein, the term “modified cell(s)” refers to one or more cell that has been surface modified with PEG and/or a second biocompatible and biodegradable hydrophobic polymer. PEG and/or biocompatible and biodegradable hydrophobic polymer blocks can be attached to a cell via a variety of conjugation linkages, non-limiting examples of which include linkage to amine, carboxyl and cysteine groups found on a cell surface (e.g., amine, carboxyl and/or cysteine groups exposed on the cell surface in membrane proteins or glycoproteins).

The term “pre-functionalized” is used herein to denote biocompatible and biodegradable hydrophobic polymer blocks or PEG polymers to which a targeting agent has been attached. In various aspects of the invention, pre-functionalized PEG and/or pre-functionalized polymer blocks are functionalized with a targeting moiety (agent). In some embodiments, the targeting moiety (agent) may be covalently bound to PEG via its a terminus and PEG can be bound to a cell via its ω terminus. A second biocompatible and biodegradable hydrophobic polymer block (e.g., polyesters) utilized in the manufacture of the disclosed modified cells can contain functional groups that react with a cell via membrane proteins or glycoproteins exposed on the cell surface. Non-limiting examples of such functional groups include, and are not limited to, amines, hydroxyl groups, carboxylic acid groups and NHS groups. Groups with which the second biocompatible and biodegradable hydrophobic polymer block functional groups react at the ω terminus of the poly(ethylene glycol) polymer are provided in the following table.

| | Reactive group on cell |
|---|---|
| PEG or Polyester functional group | surface or targeting agent |
| N-hydroxysuccinimide (NHS) | Amine |
| Amine | Hydroxyl, Carboxylic acid |
| Hydroxyl | Amine |
| Carboxylic acid | Amine |

Additionally, biocompatible and biodegradable hydrophobic polymer blocks (sometimes referred to as polyester(s) herein) can be functionalized (modified) to contain a targeting agent and attached to a cell surface. As noted in the table provided above, functional groups on the polyester(s) can react with amine, hydroxyl, or carboxylic acid groups on the targeting moiety and/or the surface of the cell to be modified. Thus, in some embodiments of the invention, a modified cell can produced that has pre-functionalized PEG, pre-functionalized polyesters, or both pre-functionalized PEG and pre-functionalized polyesters on the cell surface. Other embodiments provide cells that have been modified to have PEG, polyesters, or both PEG and polyesters on the cell surface that are then functionalized with a targeting moiety.

Targeting agents disclosed herein can contain, or be modified to contain, a functional group that can be reacted with the α terminus of a polymer (e.g., PEG) in order to produce a polymer conjugated to a targeting moiety. The functional groups include any moiety that can be used to create a covalent bond with a polymer (e.g., PEG), such as amino, hydroxy, and thio. For example, targeting agents can be can be substituted with NH₂, SH or OH, which are either bound directly to the targeting agent or via an additional group, e.g., alkyl or phenyl. In a non-limiting example, aniline, alkyl-NH₂ (e.g., (CH₂)₁₋₆NH₂), or alkyl-SH (e.g., (CH₂)₁₋₆NH₂) can be used to link the targeting agent to a polymer via the free NH₂ and SH groups to form a covalent bond.

The conjugation of a functionalized PEG polymer (a PEG polymer comprising one or more targeting agents at its α terminus and reactive functional groups at the ω terminus) and a second biocompatible and biodegradable hydrophobic polymer can be performed according to methods known in the art via functional groups at the ω terminus of a functionalized PEG polymer and reactive groups present in the second biocompatible and biodegradable hydrophobic polymer. For example, EDC-NHS chemistry (1-ethyl-3-(3-dimethylaminopropyl) carbodiimide hydrochloride and N-hydroxysuccinimide) or a reaction involving a maleimide or a carboxylic acid can be used. The conjugation of a poly(ester) and a poly(ether) to form a poly(ester-ether), can be performed in an organic solvent, such as, but not limited to, dichloromethane, acetonitrile, chloroform, dimethylformamide, tetrahydrofuran, acetone, or the like. Specific reaction conditions can be determined by those of ordinary skill in the art using no more than routine experimentation.

In another set of embodiments, a conjugation reaction may be performed by reacting a polymer that comprises a carboxylic acid functional group (e.g., a poly(ester-ether) compound) with a polymer or other moiety (such as a targeting moiety) comprising an amine. For instance, a targeting moiety, such as a low-molecular weight PSMA ligand, a peptide hormone or an antibody, may be reacted with an amine to form an amine-containing moiety, which can then be conjugated to the carboxylic acid of the polymer. Such a reaction may occur as a single-step reaction, i.e., the conjugation is performed without using intermediates such as N-hydroxysuccinimide or a maleimide. The conjugation reaction between the amine-containing moiety and the carboxylic acid-terminated polymer (such as a poly(ester-ether) compound) may be achieved, in one set of embodiments, by adding the amine-containing moiety, solubilized in an organic solvent such as (but not limited to) dichloromethane, acetonitrile, chloroform, tetrahydrofuran, acetone, formamide, dimethylformamide, pyridines, dioxane, or dimethysulfoxide, to a solution containing the carboxylic acid-terminated polymer. The carboxylic acid-terminated polymer may be contained within an organic solvent such as, but not limited to, dichloromethane, acetonitrile, chloroform, dimethylformamide, tetrahydrofuran, or acetone. Reaction between the amine-containing moiety and the carboxylic acid-terminated polymer may occur spontaneously, in some cases. Unconjugated reactants may be washed away after such reactions, and the polymer may be precipitated in solvents such as, for instance, ethyl ether, hexane, methanol, or ethanol.

The subject application also provides for the use of derivatives of PEG (e.g., analogs where functional groups, such as carboxylic acids, are protected) in the synthesis of the poly(ethylene glycol) polymer to which targeting agents are attached that improve its solubility and avoid potential side reactions. After the targeting agent is coupled to the PEG polymer at the α terminus, deprotection of the protected functional groups can be performed to regain the original chemically active functional group (e.g., a carboxylic acid).

Examples of second biocompatible and biodegradable hydrophobic polymer blocks that can be used in the manufacture of the claimed modified cells can be polyesters. Exemplary polyesters suitable for use in the manufacture of the disclosed modified cells include copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEGylated polymers and copolymers of lactide and glycolide (e.g., PEGylated PLA, PEGylated PGA, PEGylated PLGA, and derivatives thereof. In some embodiments, polyesters include, for example, polyanhydrides, poly(ortho ester) PEGylated poly(ortho ester), poly(caprolactone), PEGylated poly(caprolactone), polylysine, PEGylated polylysine, poly(ethylene inline), PEGylated poly(ethylene imine), poly(L-lactide-co-L-lysine), poly(serine ester), poly(4-hydroxy-L-proline ester), poly[a-(4-aminobutyl)-L-glycolic acid], and derivatives thereof.

As discussed above, the polymer may be PLGA in some embodiments. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid-glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

PEG suitable for use in the surface modification of cells can have a molecular weight of 1,000-20,000, e.g., 5,000-10,000, 5,000-20,000, e.g., 10,000-20,000. PLA polymers suitable for use in the surface modification of cells can have a molecular weight of 5,000-100,000 (e.g., 15,000-45,000, 20,000-70,000, or 20,000-50,000) and PLGA polymers suitable for use in the surface modification of cells can have a molecular weight of 5,000-100,000 (e.g., 15,000-45,000, 20,000-70,000 or 20,000-50,000).

The modified cells disclosed herein can be used for the treatment of various diseases and disorders within a subject. A subject may be a human or non-human animal. Examples of subjects include, but are not limited to, a mammal such as a dog, a cat, a horse, a donkey, a rabbit, a cow, a pig, a sheep, a goat, a rat, a mouse, a guinea pig, a hamster, a primate, a human or the like.

### Bioactive Moieties

### Therapeutic Agents

As discussed above, modified cells provided by the subject invention provide therapeutic agents (bioactive moieties) suitable for the treatment of various diseases and/or disorders in a subject. In various aspects of the invention, modified cells are genetically engineered to secrete one or more desired therapeutic agents. Non-limiting examples of therapeutic agents include, and are not limited to, protein or peptide based therapeutic agents. Some exemplary therapeutic agents include cytokines, chemokines, lymphokines, antibodies, insulin, antigens, hormones, and various combinations thereof. Thus, modified cells provided by this disclosure can be genetically engineered to express one or more of the following: IL-1α, IL-1β, IL-2, IL-3, IL-4, IL-5, IL-6, IL-7, IL-8, IL-9, IL-10, IL-11, IL-12elasti, IL-13, IL-15, I1-16, I1-18, IL-18 Bpa, IL-23, IL-24, VIP, erythropoietin, GM-CSF, CD40 ligand (CD40L or CD154), G-CSF, M-CSF, platelet derived growth factor, TNF-α; TNF-β; MSF, FLT-3 ligand, EGF, fibroblast growth factor; aFGF; bFGF; FGF-3; FGF-4; FGF-5; FGF-6; FGF-7; insulin-like growth factor 1; IGF-2; vascular endothelial growth factor; IFN-γ; IFN-α; IFN-β; nerve growth factor; leukemia inhibitory factor; ciliary neurotrophic factor; oncostatin M; stem cell factor; TGF-α; TGF-β1; TGF-β2; LIGHT/TNFSF14; sTALL-1/TNFSF13B, TWEAK or a chemokine selected from the group consisting of BCA-1/BLC-1, BRAK/Kec, CXCL16, CXCR3, ENA-78/LIX, Eotaxin-1, Eotaxin-2/MPIF-2, Exodus-2/SLC, Fractalkine/Neurotactin, GROalpha/MGSA, HCC-1, I-TAC, Lymphotactin/ATAC/SCM, MCP-1/MCAF, MCP-3, MCP-4, MDC/STCP-1/ABCD-1, MIP-1α, MIP-1β, MIP-2α/GROβ, MIP-3α/Exodus/LARC, MIP-3β/Exodus-3/ELC, MIP-4/PARC/DC-CK1, PF-4, RANTES, SDF1α, TARC, and TECK. In certain embodiments, the modified cells provided herein are transformed with genes encoding CD40L and GM-CSF and targeted to tumor or cancer sites using antibodies specific for the tumor.

In another embodiment, modified cells provided by the subject invention may also be used to for cell transplantation.

For the purposes of this invention, genetically engineered modified cells can be co-administered with allogeneic cancer cell lines, autologous or heterologous tumor cells, autologous or heterologous cancer cells, or autologous or heterologous malignant cells. In some embodiments, the allogeneic cancer cell lines, autologous tumor cells, cancer cells, or malignant cells can be induced to undergo apoptosis according to methods well known in the art. Such methods include, and are not limited to, contacting autologous tumor cells, cancer cells, malignant cells, or one or more allogeneic cancer cell line, with radiation, antibodies to the Fas antigen, non-steroidal anti-inflammatory compounds (such as aspirin and salicylate), and/or chemotherapeutic agents or glucocorticoids, such as chlorambucil, 2-chloro-28-deoxyadenosine, fludarabine, camptothecin, and mitoxantrone.

When a composition comprising genetically engineered modified cells and allogeneic cancer cell lines, autologous tumor cells, cancer cells, or malignant cells are administered to a subject, an adjuvant and may, optionally, be used. Compositions may also be formulated in any carriers, including for example, carriers described in E. W. Martin's Remington's Pharmaceutical Science, Mack Publishing Company, Easton, Pa. Any of a variety of adjuvants may be employed in the compositions and vaccines of this invention. Non-limiting examples of adjuvants include AlK(SO₄)₂; AlNa(SO₄)₂; AlNH₄(SO₄); silica; alum; Al(OH)₃; Ca₃(PO₄)₂; kaolin; mineral oil; lipid A; Bordatella pertussis antigens or extracts; or Mycobacterium tuberculosis antigens or extracts. Other adjuvants are commercially available and include, for example Freund's Incomplete or Complete adjuvant, Merck Adjuvant 65 (Merck and Company, Inc., Rahway, N.J.), alum, biodegradable microspheres, monophosphoryl lipid A and quil A.

The subject invention also provides methods of providing tumor antigens or cancer antigens to antigen presenting cells (e.g., dendritic cells) and/or methods of activating the immune system of (or inducing an immune response in) an individual comprising the administration of a composition comprising: 1) apoptotic, autologous tumor, cancerous, carcinoma, malignant cells, or one or more types of allogeneic cells to an individual; and 2) genetically engineered modified cells containing polynucleotides encoding GM-CSF and CD40 ligand. The genetically engineered modified cells express GM-CSF and CD40L in amounts sufficient to activate antigen presenting cells or the immune system of the individual.

In yet another embodiment, the composition can contain the genetically engineered modified cells of the instant invention and various combinations of apoptotic: 1) autologous tumor cells, 2) cancerous cells, 3) carcinoma cells, 4) malignant cells, and 5) one or more types of allogeneic cells. The composition can, optionally, also contain an adjuvant. In some embodiments, methods of treating a cancer in a subject may further comprise the administration of chemotherapeutic agents or radiation treatment to the individual. The administration of chemotherapeutic agents or radiation to the individual can occur prior to, concurrent to, or subsequent to the administration of a composition as disclosed in this paragraph.

In another embodiment, the subject invention also provides methods of providing cells which target they lymph nodes comprising the administration of a composition comprising: 1) immune cells, such as lymphocytes; and wherein the immune cells are modified to target the lymph node. One example of a ligand is L-selectin.

Another aspect of the invention provides for the treatment of tumors, carcinomas, malignancies, or cancers in individuals comprising the administration of a composition comprising a therapeutically effective amount of GM-CSF/CD40L expressing modified cell or cell line and autologous tumor cells, cancer cells, allogeneic cell lines, malignant cells, or various combinations thereof that have been induced to undergo apoptosis. The phrase “therapeutically effective amount” is intended to convey that the amount of GM-CSF and CD40L expressed by said cell or cell line is sufficient to induce (or activate) antigen presenting cells found in the treated individual and mediate a therapeutic effect, including, but not limited to a reduction in tumor/cancer burden, a decrease in metastasis or reduced invasiveness of a particular type of cancer or tumor. The composition of apoptotic cells and GM-CSF/CD40L expressing cells can be administered to an individual according to methods well known in the art. In various embodiments, the compositions of the invention can be administered orally, parenterally, as sprays (including inhalation sprays), topically, rectally, nasally, buccally, vaginally or via an implanted reservoir. The term parenteral as used herein includes subcutaneous, intradermal, intravenous, intrastriatial, intramuscular, intraperitoneal, intrathecal, intraventricular, infrasternal or intracranial injection and infusion techniques. Other embodiments provide for the administration of said composition into a tumor mass or a locus of metastasis.

Examples of tumors, cancers, carcinomas, and malignancies suitable for treatment according to the subject invention include, and are not limited to, ACTH-producing tumors, acute lymphocytic leukemia, acute nonlymphocytic leukemia, adrenal cortex cancers, bladder cancer, bone cancers, brain cancer, breast cancer, cervical cancer, chronic lymphocytic leukemia, chronic myelocytic leukemia, colorectal cancer, cutaneous T-cell lymphoma, endometrial cancer, esophageal cancer, Ewing's sarcoma, extrahepatic duct cancer, eye cancer, gallbladder cancer, gastric neoplasms, hairy cell leukemia, head, neck and thyroid cancer, Hodgkin's lymphoma, Kaposi's sarcoma, kidney cancer, liver cancer, lung cancer (small and/or non-small cell), malignant peritoneal effusion, malignant pleural effusion, melanoma, mesothelioma, multiple myeloma, neuroblastoma, non-Hodgkin's lymphoma, ocular admexa cancers, osteosarcoma, ovary cancer, germ cell cancer, pancreatic cancer, prostate cancer, retinoblastoma, skin cancer (basal cell carcinoma), soft-tissue sarcoma, squamous cell carcinomas, stomach cancer, testicular cancer, thyroid cancer, trophoblastic neoplasms, urologic cancer, uterine cancer, vaginal cancer, cancer of the vulva, and Wilm's tumor.

A “therapeutically effective amount” refers to an amount of modified cells that are necessary to treat or prevent the particular disease or disorder. For example, the administration of modified cells, as modified cells disclosed herein, can be used to inhibit tumor growth, inhibit or reduce proliferation, invasiveness, or metastasis of tumor or cancer cells, slow the growth of tumors or cancers or reduce the size of tumors.

### TARGETING AGENTS

As noted above, the disclosed modified cells also incorporate one or more targeting agent via the functionalized PEG and/or polymer moieties. Suitable targeting agents include, for example, antibodies and polypeptides that bind to polypeptides that are commonly over-expressed by tumor or cancer cells. Non-limiting examples of such over-expressed polypeptides are epidermal growth factor receptor (EGFR), somatostatin receptor (SSTR), insulin-like growth factor receptor, folic acid-receptor, HER2 receptor, interleukin-13 receptor, gastrin-releasing peptide receptor, CD30, vasoactive intestinal peptide receptor, gastrin receptor, prostate-specific antigen, and the estrogen receptor. Thus, suitable targeting agents for attachment to PEG and/or polyesters attached to the surface of a modified cells are human monoclonal, murine monoclonal, humanized or chimeric antibodies that bind to epidermal growth factor receptor (EGFR), somatostatin receptor (SSTR), insulin-like growth factor receptor, folic acid-receptor, HER2 receptor, interleukin-13 receptor, gastrin-releasing peptide receptor, CD30, vasoactive intestinal peptide receptor, gastrin receptor, prostate-specific antigen, and/or the estrogen receptor.

Another example of a targeting agents suitable for coupling to modified cells disclosed herein are small molecule ligands. Small molecule ligands can be used to target cancers that express particular target proteins (e.g., epidermal growth factor receptor (EGFR), somatostatin receptor (SSTR), insulin-like growth factor receptor, folic acid-receptor, HER2 receptor, interleukin-13 receptor, gastrin-releasing peptide receptor, CD30, vasoactive intestinal peptide receptor, gastrin receptor, prostate-specific antigen, and/or the estrogen receptor). Thus, polyesters and or PEG polymers disclosed herein can be functionalized with ligands such as PSMA ligands (discussed more fully below), estrogen, EGF, somatostatin, insulin-like growth factor, folic acid, heregulin, IL-13, CD30 ligand, vasoactive intestinal peptide, gastrin-releasing peptide, and/or gastrin and modified cells targeted to specific sites using such ligands.

The modified cells produced in accordance with the disclosed methods can be administered alone or in a composition. When administered as a composition, the composition can be a pharmaceutical (e.g., physiologically acceptable) composition. The composition comprises a carrier (e.g., a pharmaceutically or physiologically acceptable carrier) and the modified cells. Any suitable carrier (e.g., water, saline, and PBS) can be used within the context of the invention, and such carriers are well known in the art. The choice of carrier will be determined, in part, by the particular site to which the composition is to be administered and the particular method used to administer the composition. Suitable carriers, as well as other components suitable for use in the composition of the invention, are known in the art (e.g., Remington's Pharmaceutical Sciences, 17th ed., (Mack Publishing Company, Philadelphia, Pa., 1985)). Additionally, the composition can comprise additional active agents, such as anti-cancer/chemotherapeutic agents.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A method of preparing a modified cell comprising:
providing a targeting agent;
providing a first polymer comprising a functionalized poly(ethylene glycol) (PEG) polymer and, optionally, a second polymer that contains at least one functional group selected from a hydroxyl group, a NHS group or an amine group that reacts the functional group present on the surface of the cell;
reacting the functionalized poly(ethylene glycol) polymer and/or second polymer with the targeting agent to form a targeting agent-PEG polymer complex and/or a targeting agent-second polymer complex; and
reacting the targeting agent-PEG polymer complex and/or targeting agent-second polymer complex with a cell to form a modified cell to which said complex or complexes are covalently attached.

2. The method of claim 1, wherein the poly(ethylene glycol) is hetero-bifunctional and said targeting agent is covalently bound to the α terminus of said poly(ethylene glycol) and at least one functional group is present on the ω terminus of said poly(ethylene glycol) to form an attachment point with a cell.

3. The method of claim 2, wherein the ω terminus of said poly(ethylene glycol) contains a hydroxyl (—OH) group or an amine (—NH₂) group at the free ω terminus.

4. The method of claim 1, wherein a second polymer is reacted with said cell and said second polymer comprises a reactive group that reacts with a group on the surface of said cell and, optionally, has been pre-functionalized with one or more targeting agent.

5. The method of claim 1, wherein the second polymer is a polyester copolymer that contains at least one functional group selected from a hydroxyl group, a NHS group or an amine group and that reacts the functional group present on the surface of the cell.

6. The method of claim 5, wherein said polyester copolymer comprises a heteropolymer or a homopolymer.

7. The method of claim 5, wherein said heteropolymer comprises lactic acid and glycolic acid units or poly(lactic acid-co-glycolic acid) and poly(lactide-co-glycolide) units (PLGA); and said homopolymer comprises glycolic acid units (PGA), lactic acid units (PLA), poly-L-lactic acid units, poly-D-lactic acid units, poly-D,L-lactic acid units, poly-L-lactide units, poly-D-lactide units or poly-D,L-lactide units.

8. The method of claim 5, wherein said polyester copolymer is selected from polyhydroxyacids; PEGylated polymers and copolymers of lactide units and glycolide units, PEGylated PLA, PEGylated PGA, PEGylated PLGA, polyanhydrides, poly(ortho ester) PEGylated poly(ortho ester), poly(caprolactone), PEGylated poly(caprolactone), polylysine, PEGylated polylysine, poly(ethylene inline), PEGylated poly(ethylene imine), poly(L-lactide-co-L-lysine), poly(serine ester), poly(4-hydroxy-L-proline ester), poly[a-(4-aminobutyl)-L-glycolic acid] or derivatives thereof.

9. The method of claim 1, wherein free carboxylic acid groups or free hydroxyl groups on said targeting agent are protected prior to reacting the functionalized poly(ethylene glycol) polymer with the targeting agent to form a targeting agent-PEG polymer complex.

10. The method of claim 1, wherein said cell has been genetically modified to express a therapeutic agent or an antigen.

11. The method of claim 5, wherein said functional group of said second polymer or said copolymer is an amine group that reacts with a hydroxyl group or carboxylic acid group on said targeting agent-PEG complex.

12. The method of claim 5, wherein said functional group of said second polymer or said copolymer is a NHS or hydroxyl group that reacts with an amine group on said targeting agent-PEG complex.

13. The method of claim 1, wherein said second polymer is a blend of at least two polymers which can be the same or different polymer, wherein the first of said at least two polymers contains at least one hydroxyl group or an NHS group as a functional group and the second of said at least two polymers contains at least one amine group as said functional group.

14. The method of claim 1, wherein said cells are genetically modified to express a therapeutic agent selected from one or more of the following: IL-1α, IL-1β, IL-2, IL-3, IL-4, IL-5, IL-6, IL-7, IL-8, IL-9, IL-10, IL-11, IL-12elasti, IL-13, IL-15, I1-16, I1-18, IL-18Bpa, IL-23, IL-24, VIP, erythropoietin, GM-CSF, CD40 ligand (CD40L or CD154), G-CSF, M-CSF, platelet derived growth factor, TNF-α; TNF-β; MSF, FLT-3 ligand, EGF, fibroblast growth factor; aFGF; bFGF; FGF-3; FGF-4; FGF-5; FGF-6; FGF-7; insulin-like growth factor 1; IGF-2; vascular endothelial growth factor; IFN-γ; IFN-α; IFN-β; nerve growth factor; leukemia inhibitory factor; ciliary neurotrophic factor; oncostatin M; stem cell factor; TGF-α; TGF-β1; TGF-β2; LIGHT/TNFSF14; sTALL-1/TNFSF13B, TWEAK or a chemokine selected from the group consisting of BCA-1/BLC-1, BRAK/Kec, CXCL16, CXCR3, ENA-78/LIX, Eotaxin-1, Eotaxin-2/MPIF-2, Exodus-2/SLC, Fractalkine/Neurotactin, GROalpha/MGSA, HCC-1, I-TAC, Lymphotactin/ATAC/SCM, MCP-1/MCAF, MCP-3, MCP-4, MDC/STCP-1/ABCD-1, MIP-1α, MIP-1β, MIP-2α/GROG, MIP-3α/Exodus/LARC, MIP-3β/Exodus-3/ELC, MIP-4/PARC/DC-CK1, PF-4, RANTES, SDF1α, TARC and TECK.

15. The method of claim 14, wherein said genetically modified cells express CD40L and GM-CSF.

16. The method of claim 1, wherein said at least one targeting agent is selected from human monoclonal, murine monoclonal, humanized or chimeric antibodies that bind to epidermal growth factor receptor (EGFR), somatostatin receptor (SSTR), insulin-like growth factor receptor, folic acid-receptor, HER2 receptor, interleukin-13 receptor, gastrin-releasing peptide receptor, CD30, vasoactive intestinal peptide receptor, gastrin receptor, prostate-specific antigen, and/or the estrogen receptor.

17. The method of claim 1, wherein said at least one targeting agent is selected from PSMA ligands, estrogen, EGF, somatostatin, insulin-like growth factor, folic acid, heregulin, IL-13, CD30 ligand, vasoactive intestinal peptide, gastrin-releasing peptide, and/or gastrin.

18. The method of claim 1, further comprising formulating said modified cell in a pharmaceutically acceptable excipient.

19. A modified cell produced according to the methods of claim 1.

20. A composition comprising the modified cell of claim 20 and a pharmaceutically acceptable carrier.

21. The composition of claim 20, wherein said composition further comprises allogeneic cancer cell lines, autologous or heterologous tumor cells, autologous or heterologous cancer cells, or autologous or heterologous malignant cells.

22. A method of treating a disease comprising the administration of a composition according to claim 20 to a subject in an amount sufficient to treat said disease.

23. The method of claim 22, wherein said disease is selected from ACTH-producing tumors, acute lymphocytic leukemia, acute nonlymphocytic leukemia, adrenal cortex cancers, bladder cancer, bone cancers, brain cancer, breast cancer, cervical cancer, chronic lymphocytic leukemia, chronic myelocytic leukemia, colorectal cancer, cutaneous T-cell lymphoma, endometrial cancer, esophageal cancer, Ewing's sarcoma, extrahepatic duct cancer, eye cancer, gallbladder cancer, gastric neoplasms, hairy cell leukemia, head, neck and thyroid cancer, Hodgkin's lymphoma, Kaposi's sarcoma, kidney cancer, liver cancer, small cell lung cancer, non-small cell lung cancer, malignant peritoneal effusion, malignant pleural effusion, melanoma, mesothelioma, multiple myeloma, neuroblastoma, non-Hodgkin's lymphoma, ocular admexa cancers, osteosarcoma, ovary cancer, germ cell cancer, pancreatic cancer, prostate cancer, retinoblastoma, basal cell carcinoma, soft-tissue sarcoma, squamous cell carcinomas, stomach cancer, testicular cancer, thyroid cancer, trophoblastic neoplasms, urologic cancer, uterine cancer, vaginal cancer, cancer of the vulva, and Wilm's tumor.

24. The method of claim 23, further comprising the administration of a chemotherapeutic agent to said subject or the administration of a radiation treatment to said subject.
